(19) United States
(12) Patent Application Publication
(10) Pub. No.: US 2009/0017543 A1
(43) Pub. Date: Jan. 15, 2009

(54) Viral Vectors

(76) Inventors: Fraser Wilkes, Abingdon (GB); James Miskin, The Oxford Science Park (GB); Kyriacos Mitrophanous, The Oxford Science Park (GB); Susan Kingsman, The Oxford Science Park (GB)

Correspondence Address: FROMMER LAWRENCE & HAUG, 745 FIFTH AVENUE- 10TH FL., NEW YORK, NY, 10151, US

(21) Appl. No.: 12/138,993

(22) Filed: Jun. 13, 2008

Related U.S. Application Data
(63) Continuation-in-part of application No. PCT/GB2006/004811, filed on Dec. 20, 2006.

(30) Foreign Application Priority Data
Dec. 22, 2005 (GB) 0526211.8

(51) Int. Cl. C12N 15/86; C12N 15/63

(52) U.S. Cl. 435/456; 435/320.1

(57) ABSTRACT

The present invention relates to an integration defective retroviral vector particle for gene therapy comprising a viral genome, wherein said vector particle is capable of infecting a mammalian target cell.

### CROSS-REFERENCE TO RELATED APPLICATIONS

This application is a continuation-in-part of International Application No. PCT/GB2006/004811, filed Dec. 20, 2006, published as WO 2007/071994 on Jun. 28, 2007, and claiming priority to British Application No. GB 0526211.8, filed Dec. 22, 2005.

All of the foregoing applications, as well as all documents cited in the foregoing applications (“application documents”) and all documents cited or referenced in the application documents are incorporated herein by reference. Also, all documents cited in this application (“herein-cited documents”) and all documents cited or referenced in herein-cited documents are incorporated herein by reference. In addition, any manufacturer's instructions or catalogues for any products cited or mentioned in each of the application documents or herein-cited documents are incorporated by reference. Documents incorporated by reference into this text or any teachings therein can be used in the practice of this invention. Documents incorporated by reference into this text are not admitted to be prior art.

### FIELD OF THE INVENTION

The present invention relates to integration defective viral vectors and their use in gene therapy.

### BACKGROUND OF THE INVENTION

The success of gene therapy techniques depends largely on the ability to achieve regulated expression of transferred genes in a manner safe to humans. In recent years, retroviruses have been proposed as delivery vehicles for use in gene therapy. A particularly significant feature of retroviruses is their replicative strategy which includes reverse transcription of viral RNA into linear double stranded DNA and subsequent integration of this DNA into the genome of a host cell.

During the process of infection, a retrovirus initially attaches to a specific cell surface receptor. On entry into the susceptible host cell, the retroviral RNA genome is then copied to DNA by the virally encoded reverse transcriptase which is carried inside the parent virus. This DNA is transported to the host cell nucleus where it subsequently integrates into the host genome. At this stage, it is typically referred to as the provirus. The provirus is stable in the host chromosome during cell division and is transcribed like other cellular genes. The provirus encodes the proteins and packaging machinery required to make more virus, which can leave the cell by a process sometimes called “budding”.

Each virus comprises genes called gag, pol and env which code for virion proteins and enzymes. In the provirus, the retroviral genome is flanked at both ends by regions called long terminal repeats (LTRs). The LTRs are responsible for proviral integration and transcription. They also serve as enhancer-promoter sequences. In other words, the LTRs can control the expression of the viral genes. Encapsidation of the retroviral RNAs occurs by virtue of a packaging (psi) sequence located at the 5′ end of the genome.

In a typical recombinant retroviral vector for use in gene therapy, at least part of one or more of the gag, pol and env protein coding regions may be removed from the virus. This makes the retroviral vector replication-defective. The removed portions may be replaced by a nucleotide sequence of interest (NOI) in order to generate a virus capable of integrating its genome into a host genome but wherein the modified viral genome is unable to propagate itself due to a lack of structural proteins. When integrated in the host genome, expression of the NOI may occur—resulting in, for example, a therapeutic and/or a diagnostic effect.

Limiting gene expression may also be desirable if the transduced gene product is toxic to the host and in therapies where short-term expression of the transduced gene product is desirable. This has been difficult to achieve to date because of a lack of suitable vectors and transfection methods. Particularly, there has been a lack of suitable vectors and transfection methods for RNA, which have yet to achieve satisfactory levels of transfer in vivo.

In retroviral and other recombination-based approaches, a second potential concern arises in the unpredictability of where the new DNA inserts into the chromosomes of transfected cells resulting in the inactivation or altered transcriptional regulation of host cell genes.

Integration defective vectors have been reported. However these vectors generally only contain a single mutation in the integrase protein and may retain some residual integrase activity.

Accordingly, there exists a significant need in the art for effective gene therapy methods which reduce unwanted immune responses, allow for short-term gene expression and have improved safety. The present invention addresses these needs.

### SUMMARY OF THE INVENTION

Retroviral and lentiviral constructs are disclosed which are lacking or disabled in key proteins/sequences so as to prevent integration of the retroviral or lentiviral genome into the target cell genome. In particular, we show that viral constructs lacking each of the amino acids making up the highly conserved DDE motif (Engelman and Craigie (1992) J. Virol. 66:6361-6369; Johnson et al. (1986) Proc. Natl. Acad. Sci. USA 83:7648-7652; Khan et al. (1991) Nucleic Acids Res. 19:851-860) of retroviral integrase enables the production of integration defective vectors useful in gene therapy. Retroviral and lentiviral constructs disabled in key proteins/sequences so as to prevent reverse transcription of RNA to DNA are also disclosed. The constructs of the present invention have particular use in the delivery of therapeutic RNAs. By therapeutic RNA it is meant a sequence which functions at the RNA level such as an RNA that does not require integration and/or reverse transcription to have a therapeutic effect.

According to one aspect of the present invention there is provided an integration defective retroviral vector particle for gene therapy comprising a viral genome, wherein said vector particle is capable of infecting a mammalian target cell.

According to another aspect of the present invention there is provided an integration defective lentiviral vector particle for gene therapy comprising a viral genome, wherein said vector particle is capable of infecting a mammalian target cell.

Preferably the viral genome comprises a nucleotide sequence of interest (NOI).

Preferably the vector particle comprises a disabled integrase protein.

In a particularly preferred embodiment the DDE motif of the integrase is removed in its entirety. Put another way, none of the D, D or E amino acids making up the motif are present in the integrase protein. This may be achieved by removing or replacing each of the DDE amino acids of the motif. In one embodiment each of the D, D and E amino acids of the DDE motif are replaced with a different amino acid. In another embodiment each of the DDE amino acids are removed from the integrase protein.

In a particularly preferred embodiment each of the D, D and E amino acids of the DDE motif are replaced with different amino acids or are absent from the integrase protein. Put another way, none of the D, D or E amino acids making up the motif are present in the integrase protein.

In one embodiment the vector particle does not comprise an integrase protein.

According to another aspect of the present invention there is provided a retroviral vector particle for gene therapy, the vector particle being capable of infecting mammalian target cells, wherein the viral genome is incapable of undergoing reverse transcription following infection of a cell by the retroviral vector particle.

According to another aspect of the present invention there is provided a lentiviral vector particle for gene therapy, the vector particle being capable of infecting mammalian target cells, wherein the viral genome is incapable of undergoing reverse transcription following infection of a cell by the lentiviral vector particle.

Preferably the vector particle is incapable of undergoing integration.

Preferably the viral genome comprises a nucleotide sequence of interest (NOI).

The vector particle of the present invention may comprise a disabled reverse transcriptase protein, or may not comprise a reverse transcriptase protein.

The vector particle of the present invention may comprise one or more disabled pol proteins, or may not comprise one or more pol proteins

Preferably the NOI is a therapeutic RNA.

Preferably the therapeutic RNA is selected from the group comprising mRNA, siRNA, shRNA micro-RNA, ribozyme, antisense RNA and tRNA.

In a particularly preferred embodiment the NOI is a siRNA.

Preferably the viral genome comprises a disabled gag, pol and/or env gene.

In one embodiment the viral genome does not comprise gag, pol and/or env gene.

Preferably the viral genome comprises a disabled gag, pol and env gene or does not comprise a gag, pol and env gene.

In one embodiment the viral genome comprises a disabled primer binding site (PBS) and/or att site.

In another embodiment the viral genome does not comprise a primer binding site (PBS) and/or att site.

Preferably one or more viral accessory genes, including rev, tat, vif, nef, vpr, vpu, vpx and S2 or functional equivalents thereof, are disabled or absent from the viral genome.

In another embodiment all of the viral accessory genes are disabled or absent from the viral genome.

Preferably the dUTPase gene is disabled or absent from the viral genome.

In another embodiment the viral genome comprises a packaging sequence.

In another embodiment the retroviral vector particle of the present invention comprises a viral genome which is free of retroviral RNA sequence with the proviso that a packaging sequence is present.

In another embodiment the lentiviral vector particle of the present invention comprises a viral genome which is free of lentiviral RNA sequence with the proviso that a packaging sequence is present.

The packaging sequence may be an extended packaging sequence.

In another embodiment the viral genome consists of a NOI and a packaging sequence.

Preferably the lentiviral vector particle of the present invention is derived from a non-primate lentivirus.

In one embodiment the non-primate lentivirus is EIAV.

In one embodiment one or more accessory genes selected from S2, rev and tat are disabled or absent

In another embodiment all the accessory genes S2, rev and tat are disabled or absent

According to another aspect of the present invention there is provided a vector particle production system for producing the retroviral or lentiviral vector particle of the present invention, which system comprises a set of nucleic acid sequences encoding the viral genome, gag and env proteins or a functional substitute thereof.

In one embodiment the nucleic acid sequences encode a disabled pol.

In another embodiment the entire pol gene is absent from the nucleic acid sequences.

In one embodiment the nucleic acid sequences encode a disabled integrase.

Preferably the DDE motif of the integrase protein is removed in its entirety. Put another way, none of the D, D or E amino acids making up the motif are present in the integrase protein. This may be achieved by replacing each of the D, D and E amino acids of the DDE motif with a different amino acid or by removing each of these amino acids from the integrase protein. In one embodiment each of the D, D and E amino acids of the DDE motif are replaced with a different amino acid such that the integrase protein is no longer active. In another embodiment each of the DDE amino acids are removed from the integrase protein.

In another embodiment the entire integrase gene is absent from the nucleic acid sequences.

In another embodiment the nucleic acid sequences encode a disabled reverse transcriptase.

In another embodiment the entire reverse transcriptase gene is absent from the nucleic acid sequences.

According to another aspect of the present invention there is provided a set of DNA constructs used in the system of the present invention comprising a DNA construct encoding a viral vector genome and a DNA construct encoding gag protein or a functional substitute therefore.

Preferably the DNA constructs further encode an env protein or a functional substitute thereof.

According to another aspect of the present invention there is provided a set of DNA constructs of the present invention in one or more expression vectors.

According to another aspect of the present invention there is provided a set of DNA constructs of the present invention in a host cell.

According to another aspect of the present invention there is provided a process for preparing a vector particle of the present invention comprising introducing a set of nucleic acid sequences or DNA constructs of the present invention into a host cell, and obtaining the vector particle.

According to another aspect of the present invention there is provided a pharmaceutical composition comprising a vector particle of the present invention and a pharmaceutically acceptable excipient, diluent or carrier.

According to another aspect of the present invention there is provided a target cell infected or transduced with a vector particle of the present invention.

According to another aspect of the present invention there is provided a process for preparing a viral vector particle of the present invention comprising introducing a set of nucleic acid sequences or DNA constructs of the present invention into a host cell and obtaining the vector particle.

According to another aspect of the present invention there is provided a pharmaceutical composition comprising the vector particle of the present invention.

According to another aspect of the present invention there is provided a target cell infected or transduced with a vector particle of the present invention.

According to another aspect of the present invention there is provided use of a vector particle of the present invention, a vector particle production system of the present invention or a set of DNA constructs of the present invention in the preparation of a medicament for treating viral infection, such as, but not limited to, HIV, influenza, Herpesviridae, human papillomavirus or Ebola infection.

According to another aspect of the present invention there is provided use of a vector particle of the present invention, a vector particle production system of the present invention, or a set of DNA constructs of the present invention in the preparation of a medicament for treating an intracellular infection.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

### BRIEF DESCRIPTION OF THE DRAWINGS

The following Detailed Description, given by way of example, but not intended to limit the invention to specific embodiments described, may be understood in conjunction with the accompanying drawings, incorporated herein by reference. Various preferred features and embodiments of the present invention will now be described by way of non-limiting example and with reference to the accompanying drawings in which:

FIGS. 1A and 1B shows protection from staurosporine (STS)-mediated toxicity in cortical neurons transduced with integrase defective vectors.

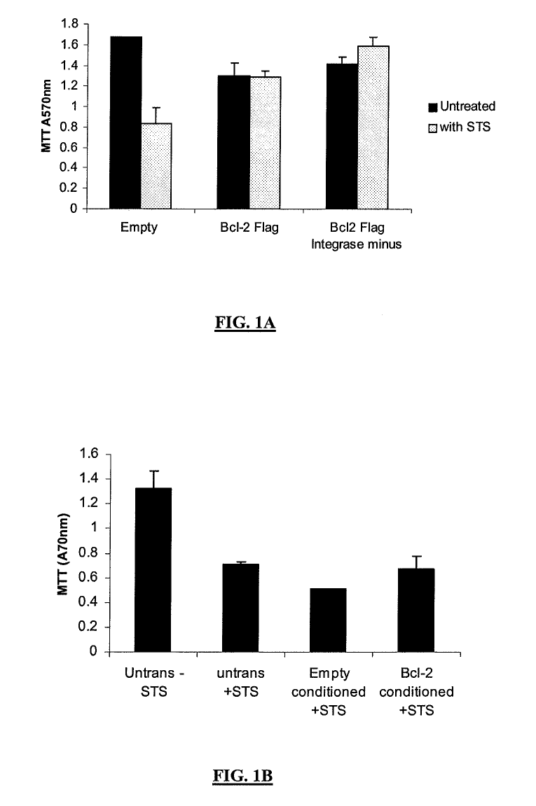

FIG. 2 shows that conditioned medium from cortical neurons transduced with BCL-2-Flag is unable to protect against staurosporine-induced apoptosis.

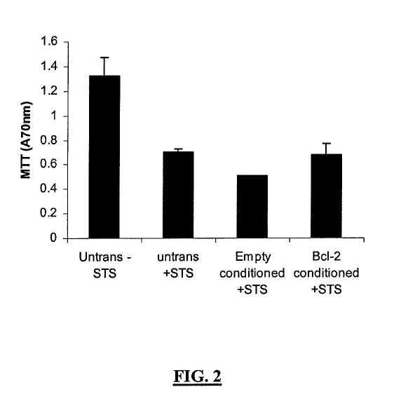

### DETAILED DESCRIPTION

Various preferred features and embodiment of the present invention will now be described by way of non-limiting example.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis (1989) Molecular Cloning: A Laboratory Manual, Second Edition, Books 1-3, Cold Spring Harbor Laboratory Press; Ausubel, F. M. et al. (1995 and periodic supplements) Current Protocols in Molecular Biology, Ch. 9, 13, and 16, John Wiley & Sons, New York, N.Y.); B. Roe, J. Crabtree, and A. Kahn (1996) DNA Isolation and Sequencing: Essential Techniques, John Wiley & Sons; J. M. Polak and James O'D. McGee (1990) In Situ Hybridization: Principles and Practice; Oxford University Press; M. J. Gait (Ed.) (1984) Oligonucleotide Synthesis: A Practical Approach, IRL Press; and, D. M. J. Lilley and J. E. Dahlberg (1992) Methods of Enzymology: DNA Structure Part A: Synthesis and Physical Analysis of DNA Methods in Enzymology, Academic Press. Each of these general texts is herein incorporated by reference.

### Polynucleotides

Polynucleotides of the invention may comprise DNA or RNA. They may be single-stranded or double-stranded. It will be understood by a skilled person that numerous different polynucleotides can encode the same polypeptide as a result of the degeneracy of the genetic code. In addition, it is to be understood that skilled persons may, using routine techniques, make nucleotide substitutions that do not affect the polypeptide sequence encoded by the polynucleotides used in the invention to reflect the codon usage of any particular host organism in which the polypeptides are to be expressed. The polynucleotides may be modified by any method available in the art. Such modifications may be carried out in order to enhance the in vivo activity or life span of the polynucleotides of the invention.

Polynucleotides such as DNA polynucleotides may be produced recombinantly, synthetically, or by any means available to those of skill in the art. They may also be cloned by standard techniques.

Longer polynucleotides will generally be produced using recombinant means, for example using PCR (polymerase chain reaction) cloning techniques. This will involve making a pair of primers (e.g. of about 15 to 30 nucleotides) flanking a region of the lipid targeting sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector.

It will be appreciated that the polynucleotide of the invention may contain only coding regions. However, it is preferred if the polynucleotide further comprises, in operable linkage, a portion of nucleic acid that allows for efficient translation of the coding sequence. It is further preferred if the polynucleotide (when in a DNA form) further comprises a promoter in operable linkage which allows for the transcription of the coding region and the portion of nucleic acid that allows for efficient translation of the coding region in a target cell.

### Protein

As used herein, the term “protein” includes single-chain polypeptide molecules as well as multiple-polypeptide complexes where individual constituent polypeptides are linked by covalent or non-covalent means. As used herein, the terms “polypeptide” and “peptide” refer to a polymer in which the monomers are amino acids and are joined together through peptide or disulfide bonds. The terms subunit and domain may also refer to polypeptides and peptides having biological function.

### Derivatives

The term “derived from” is used in its normal sense as meaning the sequence need not necessarily be obtained from a sequence but instead could be derived therefrom. By way of example, a sequence may be prepared synthetically or by use of recombinant DNA techniques.

### Disabled

The term ‘disabled’ refers to a gene or protein which is inactive, that is it has essentially no wild type activity. A gene may be disabled by preventing protein expression from the gene or by removing or modifying at least part of one or more coding regions essential for protein function. A protein may be disabled by removing or replacing one or more amino acids essential for protein function.

Preferably, the disabled gene or protein has less than 5%, 2%, or 1% of wild type activity. More preferably the disabled gene or protein has no wild type activity.

### Viruses

As it is well known in the art, a vector is a tool that allows or facilitates the transfer of an entity from one environment to another. In accordance with the present invention, and by way of example, some vectors used in recombinant DNA techniques allow entities, such as a segment of DNA (such as a heterologous DNA segment), to be transferred into a host cell for the purpose of replicating the vectors comprising a segment of DNA. Examples of vectors used in recombinant DNA techniques include but are not limited to plasmids, chromosomes, artificial chromosomes or viruses.

The retroviral vector or retroviral vector particle of the present invention may be derived from or may be derivable from any suitable retrovirus. A large number of different retroviruses have been identified. Examples include: murine leukemia virus (MLV), human T-cell leukemia virus (HTLV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV), Foamy virus (FMV). A detailed list of retroviruses may be found in Coffin et al. (1997) “Retroviruses”, Cold Spring Harbor Laboratory Press Eds: J M Coffin, S M Hughes, H E Varmus pp 758-763.

Retroviruses may be broadly divided into two categories: namely, “simple” and “complex”. Retroviruses may even be further divided into seven groups. Five of these groups represent retroviruses with oncogenic potential. A review of these retroviruses is presented in Coffin et al. (1997) (ibid).

The basic structure of retrovirus and lentivirus genomes share many common features such as a 5′ LTR and a 3′ LTR, between or within which are located a packaging signal to enable the genome to be packaged, a primer binding site, integration sites to enable integration into a host cell genome and gag, pol and env genes encoding the packaging components—these are polypeptides required for the assembly of viral particles. Integrase is encoded by the 3′ end of the pol gene, which also codes for two other viral enzymes, the protease and the reverse transcriptase. These three enzymes are initially synthesised as part of a larger polyprotein that is subsequently cleaved by the protease into the individual proteins.

Lentiviruses have additional features, such as the rev and RRE sequences in HIV, which enable the efficient export of RNA transcripts of the integrated provirus from the nucleus to the cytoplasm of an infected target cell.

In the provirus, these genes are flanked at both ends by regions called long terminal repeats (LTRs). The LTRs are responsible for proviral integration, and transcription. LTRs also serve as enhancer-promoter sequences and can control the expression of the viral genes.

The LTRs themselves are identical sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

### Reverse Transcription

Once the viral core enters the cytoplasm of the target cell, reverse transcription converts viral genomic RNA into double stranded DNA. Reverse transcriptase initiates minus-strand DNA synthesis by elongating a partially unwound primer tRNA that is hybridized to the primer binding site (PBS) in genomic RNA.

In HIV-1, tRNA^{LYS3} serves as the replication primer. Synthesis continues to the 5′ end of the genome, generating minus-strand DNA [(−)ssDNA]. As reverse transcriptase reaches the end of the template, its RNase H activity degrades the RNA strand of the RNA/DNA duplex. This allows the first strand transfer to proceed whereby (−)ssDNA is transferred to the 3′end of genome, guided by the repeat (R) sequences of the LTRs present on both ends of the RNA. Minus-strand DNA synthesis then resumes and is completed by reverse transcriptase, again accompanied by RNase H-mediated degradation of the template strand. Template digestion is incomplete and results in the generation of RNase H-resistant oligoribonucleotides rich in purines, called the polypurine tract (PPT). Plus-strand DNA synthesis is primarily at the PPT and then proceeds by copying minus-strand DNA to its 5′ end. RNase H removal of the primer tRNA facilitates the second strand transfer, in which complementary PBS segments in the plus-strand DNA and in the minus-strand DNA anneal. The plus and minus strand syntheses are then completed, with each strand serving as a template for the other. On completion of the reverse transcription, the viral DNA is translocated into the nucleus where the linear copy of the viral genome, called a pre-integration complex (PIC), is inserted into chromosomal DNA with the aid of the virion integrase to form a stable provirus. The number of possible sites of integration into the host cellular genome is very large and very widely distributed.

The term ‘incapable of undergoing reverse transcription’ used herein means the viral genome is not able to undergo reverse transcription via the conventional retroviral or lentiviral reverse transcription mechanism, such as that described above.

### Integration

Integrase first acts within the pre-integration complex by mediating an endonucleolytic cleavage at the 3′ end of each strand of viral DNA immediately beyond a conserved subterminal CA dinucleotide. This step, called 3′-processing, occurs in the cytoplasm and leaves a terminal hydroxyl group at the 3′ end of each strand of viral DNA. After the nucleoprotein complex migrates to the nucleus, integrase mediates a concerted nucleophilic attack involving the viral 3′ hydroxyl residues and phosphate residues on either side of the major groove in the target DNA, a step termed strand transfer. The two viral ends attack the target DNA in a coordinated, 5′-staggered fashion, the extent of the stagger determining the length of the virus-specific direct repeat of host DNA that flanks the integrated provirus.

Attachment (att) sites, virus-specific sequences located at each end of viral DNA, and integrase, are known to be essential for integration (Gaur et al. (1988) J. Virol. 72(6): 4678-4685). Coupled with amino acid sequence alignment, the in vitro activity data for wild-type and mutant integrase proteins have led to a working model of integrase with three domains: the amino-terminal or HHCC domain, the core or catalytic domain, and the carboxy-terminal or DNA binding domain (Gaur et al. (1988) J. Virol. 72(6):4678-4685).

The terms ‘incapable of undergoing integration’, or ‘integration defective’ used herein mean the viral genome is not able to integrate into the target cell genome via the conventional retroviral or lentiviral integration mechanism, such as that described above.

Important to the catalytic activity of the integrase is the highly conserved DDE motif found in all retroviral integrase proteins and numerous transposable elements. The DDE motif refers to three absolutely conserved acidic amino acids (two aspartic acids and one glutamic acid) in the order indicated, with a conserved spacing of generally 35 amino acids between the second and third residues (Engelman and Craigie (1992) J. Virol. 66:6361-6369; Johnson et al. (1986) Proc. Natl. Acad. Sci. USA 83:7648-7652; Khan et al. (1991) Nucleic Acids Res. 19:851-860).

In a defective retroviral or lentiviral vector genome gag, pol and env may be absent or not functional. The R regions at both ends of the RNA are repeated sequences. U5 and U3 represent unique sequences at the 5′ and 3′ ends of the RNA genome respectively.

In a typical viral vector of the present invention, at least part of one or more protein coding regions essential for replication may be removed from or disabled in the virus. This makes the viral vector replication-defective. Portions of the viral genome may also be replaced by a library encoding candidate modulating moieties operably linked to a regulatory control region and a reporter moiety in the vector genome in order to generate a vector comprising candidate modulating moieties which is capable of transducing a target non-dividing host cell and/or integrating its genome into a host genome.

A detailed list of lentiviruses may be found in Coffin et al (1997) “Retroviruses” Cold Spring Harbor Laboratory Press Eds: J M Coffin, S M Hughes, H E Varmus pp 758-763). In brief, lentiviruses can be divided into primate and non-primate groups. Examples of primate lentiviruses include but are not limited to: the human immunodeficiency virus (HIV), the causative agent of human auto-immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV) and the more recently described feline immunodeficiency virus (FIV) and bovine immunodeficiency virus (BIV).

The lentivirus family differs from retroviruses in that lentiviruses have the capability to infect both dividing and non-dividing cells (Lewis et al. (1992); Lewis and Emerman (1994)). In contrast, retroviruses, such as MLV, are unable to infect non-dividing or slowly dividing cells such as those that make up, for example, muscle, brain, lung and liver tissue.

A lentiviral or lentivirus vector, as used herein, is a vector which comprises at least one component part derivable from a lentivirus. Preferably, that component part is involved in the biological mechanisms by which the vector infects cells, expresses genes or is replicated.

The lentiviral vector may be a “non-primate” vector, i.e., derived from a virus which does not primarily infect primates, especially humans.

The non-primate lentivirus may be any member of the family of lentiviridae which does not naturally infect a primate and may include a feline immunodeficiency virus (FIV), a bovine immunodeficiency virus (BIV), a caprine arthritis encephalitis virus (CAEV), a Maedi visna virus (MVV) or an equine infectious anaemia virus (EIAV).

In one embodiment the viral vector is derived from EIAV. EIAV has the simplest genomic structure of the lentiviruses and is particularly preferred for use in the present invention. In addition to the gag, pol and env genes EIAV encodes three other genes: tat, rev, and S2. Tat acts as a transcriptional activator of the viral LTR (Derse and Newbold (1993); Maury et al. (1994)) and Rev regulates and coordinates the expression of viral genes through rev-response elements (RRE) (Martarano et al. (1994)). The mechanisms of action of these two proteins are thought to be broadly similar to the analogous mechanisms in the primate viruses (Martano et al. (ibid)). The function of S2 is unknown. In addition, an EIAV protein, Ttm, has been identified that is encoded by the first exon of tat spliced to the env coding sequence at the start of the transmembrane protein.

Preferred retroviral or lentiviral vectors of the present invention are recombinant retroviral or lentiviral vectors (recombinant viral vectors).

The term “recombinant viral vector” (RVV) refers to a vector with sufficient viral genetic information to allow packaging of an RNA genome, in the presence of packaging components, into a viral particle capable of infecting a target cell. The RVV carries non-viral coding sequences which are to be delivered by the vector to the target cell. An RVV is incapable of independent replication to produce infectious viral particles within the final target cell. Usually the RVV lacks a functional gag-pol and/or env gene and/or other genes essential for replication. The vector of the present invention may be configured as a split-intron vector. A split intron vector is described in PCT patent application WO 99/15683.

Preferably the RVV vector of the present invention has a minimal viral genome.

As used herein, the term “minimal viral genome” means that the viral vector has been manipulated so as to remove the non-essential elements and to retain the essential elements in order to provide the required functionality to infect, transduce and deliver a nucleotide sequence of interest to a target host cell. Further details of this strategy can be found in our WO 98/17815.

A minimal viral genome of the present invention may comprise (5′) R-U5-one or more nucleotide sequence of interest sequences-U3-R (3′).

In one embodiment, the minimal viral genome comprises little to no retroviral or lentiviral sequences. For example, it may only comprise a NOI (e.g., a siRNA) and a packaging signal.

However, the plasmid vector used to produce the viral genome within a host cell/packaging cell will also include transcriptional regulatory control sequences operably linked to the viral genome to direct transcription of the genome in a host cell/packaging cell. These regulatory sequences may be the natural sequences associated with the transcribed viral sequence, i.e. the 5′ U3 region, or they may be a heterologous promoter such as another viral promoter, for example the constitutive transport element (CMV) promoter. Some lentiviral genomes require additional sequences for efficient virus production. For example, in the case of HIV, rev and RRE sequence are preferably included. However the requirement for rev and RRE may be reduced or eliminated by codon optimization. Further details of this strategy can be found in our WO 01/79518. Alternative sequences which perform the same function as the rev/RRE system are also known. For example, a functional analog of the rev/RRE system is found in the Mason Pfizer monkey virus. This is known as CTE and comprises an RRE-type sequence in the genome which is believed to interact with a factor in the infected cell. The cellular factor can be thought of as a rev analog. Thus, CTE may be used as an alternative to the rev/RRE system. Any other functional equivalents which are known or become available may be relevant to the invention. For example, it is also known that the Rex protein of HTLV-I can functionally replace the Rev protein of HIV-1. It is also known that Rev and Rex have similar effects to IRE-BP.

### Packaging Sequence

As utilized within the context of the present invention the term “packaging signal” which is referred to interchangeably as “packaging sequence” or “psi” is used in reference to the non-coding, cis-acting sequence required for encapsidation of retroviral or lentiviral RNA strands during viral particle formation. In HIV-1, this sequence has been mapped to loci extending from upstream of the major splice donor site (SD) to at least the gag start codon.

As used herein, the term “extended packaging signal” or “extended packaging sequence” refers to the use of sequences around the psi sequence with further extension into the gag gene. The inclusion of these additional packaging sequences may increase the efficiency of insertion of vector RNA into viral particles. As an example, for the Murine Leukemia Virus, MoMLV, the minimum core packaging signal is encoded by the sequence (counting from the 5′ LTR cap site) from approximately nucleotide 144, up through the Pst I site (nucleotide 567). The extended packaging signal of MoMLV includes the sequence beyond nucleotide 567 up through the start of the gag/pol gene (nucleotide 621), and beyond nucleotide 1040 (Bender et al. (1987)). These sequences include about a third of the gag gene sequence.

Feline immunodeficiency virus (FIV) RNA encapsidation determinants have been shown to be discrete and non-continuous, comprising one region at the 5′ end of the genomic mRNA (R-U5) and another region that mapped within the proximal 311 nt of gag. Kaye et al. (1995) showed that mRNAs of subgenomic vectors as well as of full-length molecular clones were optimally packaged into viral particles and resulted in high-titer FIV vectors when they contained only the proximal 230 nucleotides (nt) of gag. Further 3′ truncations of gag sequences progressively diminished encapsidation and transduction. Deletion of the initial ninety 5′ nt of the gag gene abolished mRNA packaging, demonstrating that this segment is indispensable for encapsidation.

### Vector Particle Production Systems

The term ‘vector particle production system’ refers to a system comprising the necessary components for retroviral or lentiviral vector particle production.

By using producer/packaging cell lines, it is possible to propagate and isolate quantities of retroviral or lentiviral vector particles (e.g. to prepare suitable titres of the retroviral or lentiviral vector particles) for subsequent transduction of, for example, a site of interest (such as adult brain tissue). Producer cell lines are usually better for large scale production of vector particles.

As used herein, the term “packaging cell” refers to a cell which contains those elements necessary for production of infectious recombinant virus which are lacking or non-functional in the viral genome. Typically, such packaging cells contain one or more producer plasmids which are capable of expressing viral structural proteins (such as codon optimized gag-pol and env) but they typically do not contain a packaging signal.

Transient transfection has numerous advantages over the packaging cell method. In this regard, transient transfection avoids the longer time required to generate stable vector-producing cell lines and is used if the vector genome or viral packaging components are toxic to cells. If the vector genome encodes toxic genes or genes that interfere with the replication of the host cell, such as inhibitors of the cell cycle or genes that induce apoptosis, it may be difficult to generate stable vector-producing cell lines, but transient transfection can be used to produce the vector before the cells die. Also, cell lines have been developed using transient infection that produces vector titre levels that are comparable to the levels obtained from stable vector-producing cell lines (Pear et al. (1993)).

Producer cells/packaging cells can be of any suitable cell type. Producer cells are generally mammalian cells but can be, for example, insect cells.

As used herein, the term “producer cell” or “vector producing cell” refers to a cell which contains all the elements necessary for production of viral vector particles.

Preferably, the producer cell is obtainable from a stable producer cell line.

Preferably, the producer cell is obtainable from a derived stable producer cell line.

In one embodiment the packaging/producer cells of the present invention produce retroviral or lentiviral vector particles that are integration defective, in which the viral genome of the particle cannot integrate into the target cell's genome through the retroviral or lentiviral integration mechanism. In this embodiment, the packaging/producer cell is defective in a gene or sequence essential for integration. For example, the cell may comprise a disabled integrase gene, a disabled primer binding site (PBS) or a disabled att site. Preferably the entire integrase gene, PBS or att site is absent from the packaging/producer cell.

In another embodiment the packaging/producer cells of the present invention producing retroviral or lentiviral vector particles which upon infection of a target cell, do not allow for reverse transcription of the RNA genome. In this embodiment, the cell is defective in a gene or sequence essential for reverse transcription. For example, the cell may comprise a disabled reverse transcriptase gene. Preferably the entire reverse transcriptase coding region is absent from the packaging/producer cell.

Preferably the envelope protein sequences and nucleocapsid sequences are all stably integrated in the producer and/or packaging cell. However, one or more of these sequences could also exist in episomal form and gene expression could occur from the episome.

Also as discussed above, simple packaging cell lines, comprising a provirus in which the packaging signal has been deleted, have been found to lead to the rapid production of undesirable replication competent viruses through recombination. In order to improve safety, second generation cell lines have been produced wherein the 3 ′LTR of the provirus is deleted. In such cells, two recombinations would be necessary to produce a wild type virus. A further improvement involves the introduction of the gag-pol genes and the env gene on separate constructs so-called third generation packaging cell lines. These constructs are introduced sequentially to prevent recombination during transfection.

Preferably, the packaging cell lines are second generation packaging cell lines.

Preferably, the packaging cell lines are third generation packaging cell lines.

In these split-construct, third generation cell lines, a further reduction in recombination may be achieved by changing the codons. This technique, based on the redundancy of the genetic code, aims to reduce homology between the separate constructs, for example between the regions of overlap in the gag-pol and env open reading frames.

The packaging cell lines are useful for providing the gene products necessary to encapsidate and provide a membrane protein for a high titre vector particle production. The packaging cell may be a cell cultured in vitro such as a tissue culture cell line. Suitable cell lines include but are not limited to mammalian cells such as murine fibroblast derived cell lines or human cell lines. Preferably the packaging cell line is a human cell line.

Alternatively, the packaging cell may be a cell derived from the individual to be treated. The cell may be isolated from an individual and the packaging and vector components administered ex vivo followed by re-administration of the autologous packaging cells.

In more detail, the packaging cell may be an in vivo packaging cell in the body of an individual to be treated or it may be a cell cultured in vitro such as a tissue culture cell line.

In one embodiment the vector configurations of the present invention use as their production system, three transcription units expressing a genome, the gag-pol components and an envelope. The envelope expression cassette may include one of a number of envelopes such as VSV-G or various murine retrovirus envelopes such as 4070A.

### Pseudotyping

In one preferred aspect, the viral vector of the present invention has been pseudotyped. In this regard, pseudotyping can confer one or more advantages. For example, with the lentiviral vectors, the env gene product of HIV-1 based vectors would restrict these vectors to infecting only cells that express a protein called CD4. But if the env gene in these vectors has been substituted with env sequences from other RNA viruses, then they may have a broader infectious spectrum (Verma and Somia (1997)). By way of example, workers have pseudotyped an HIV-1 based vector with the glycoprotein from VSV (Verma and Somia (1997) (ibid)).

In another alternative, the Env protein may be a modified Env protein such as a mutant or engineered Env protein. Modifications may be made or selected to introduce targeting ability or to reduce toxicity or for another purpose (Valsesia-Wittman et al. (1996); Nilson et al. (1996); Fielding et al. (1998) and references cited therein).

The vector may be pseudotyped with any molecule of choice.

### VSV-G:

The envelope glycoprotein (G) of Vesicular stomatitis virus (VSV), a rhabdovirus, is another envelope protein that has been shown to be capable of pseudotyping certain retroviruses.

Its ability to pseudotype MoMLV-based retroviral vectors in the absence of any retroviral envelope proteins was first shown by Emi et al. (1991) J. Virol. 65:1202-1207). WO 94/294440 teaches that retroviral vectors may be successfully pseudotyped with VSV-G. These pseudotyped VSV-G vectors may be used to transduce a wide range of mammalian cells. Even more recently, Abe et al. (1998) J. Virol. 72 (8) 6356-6361 teach that non-infectious retroviral particles can be made infectious by the addition of VSV-G.

Burns et al. (1993) Proc. Natl. Acad. Sci. USA 90: 8033-7 successfully pseudotyped the retrovirus MLV with VSV-G and this resulted in a vector having an altered host range compared to MLV in its native form. VSV-G pseudotyped vectors have been shown to infect not only mammalian cells, but also cell lines derived from fish, reptiles and insects (Burns et al (1993) (ibid)). They have also been shown to be more efficient than traditional amphotropic envelopes for a variety of cell lines (Yee et al. (1994) Proc. Natl. Acad. Sci. USA 91:9564-9568, Emi et al. (1991) J. Virol. 65:1202-1207).

The provision of a non-retroviral pseudotyping envelope such as VSV-G protein gives the advantage that vector particles can be concentrated by ultracentrifugation to a high titre without loss of infectivity (Akkina et al. (1996) J. Virol. 70:2581-5). Retrovirus envelope proteins are apparently unable to withstand the shearing forces during ultracentrifugation, probably because they consist of two non-covalently linked subunits. The interaction between the subunits may be disrupted by the centrifugation. In comparison the VSV glycoprotein is composed of a single unit. VSV-G protein pseudotyping can therefore offer potential advantages.

### Ross River Virus

The Ross River viral envelope has been used to pseudotype a nonprimate lentiviral vector (FIV) and following systemic administration predominantly transduced the liver (Kang et al. (2002)). Efficiency was reported to be 20-fold greater than obtained with VSV-G pseudotyped vector, and caused less cytotoxicity as measured by serum levels of liver enzymes suggestive of hepatotoxicity.

Ross River Virus (RRV) is an alphavirus spread by mosquitoes which is endemic and epidemic in tropical and temperate regions of Australia. Antibody rates in normal populations in the temperate coastal zone tend to be low (6% to 15%) although sero-prevalence reaches 27 to 37% in the plains of the Murray Valley River system. In 1979 to 1980 RRV became epidemic in the Pacific Islands. The disease is not contagious between humans and is never fatal, the first symptom being joint pain with fatigue and lethargy in about half of patients (Fields Virology).

### Baculovirus GP64

The baculovirus GP64 protein has been shown to be an attractive alternative to VSVG for viral vectors used in the large-scale production of high-titer virus required for clinical and commercial applications (Kumar M, Bradow B P, Zimmerberg J (2003) Hum Gene Ther. 14 (1):67-77). Compared with VSVG, GP64 vectors have a similar broad tropism and similar native titers. Because, GP64 expression does not kill cells, 293T-based cell lines constitutively expressing GP64 can be generated.

### Rabies G

In the present invention the vector system may be pseudotyped with at least a part of a rabies G protein or a mutant, variant, homologue or fragment thereof.

Teachings on the rabies G protein, as well as mutants thereof, may be found in WO 99/61639 and well as Rose et al. (1982) J. Virol. 43: 361-364, Hanham et al. (1993) J. Virol. 67:530-542, Tuffereau et al. (1998) J. Virol. 72:1085-1091, Kucera et al. (1985) J. Virol. 55:158-162, Dietzschold et al. (1983) PNAS 80:70-74, Seif et al. (1985) J. Virol. 53:926-934, Coulon et al. (1998) J. Virol. 72:273-278, Tuffereau et al. (1998) J. Virol. 72:1085-10910, Burger et al. (1991) J. Gen. Virol. 72:359-367, Gaudin et al. (1995) J. Virol. 69:5528-5534, Benmansour et al. (1991) J. Virol. 65:4198-4203, Luo et al. (1998) Microbiol. Immunol. 42:187-193, Coll (1997) Arch. Virol. 142:2089-2097, Luo et al. (1997) Virus Res. 51:35-41, Luo et al. (1998) Microbiol. Immunol. 42:187-193, Coll (1995) Arch. Virol. 140:827-851, Tuchiya et al. (1992) Virus Res. 25:1-13, Morimoto et al. (1992) Virology 189:203-216, Gaudin et al. (1992) Virology 187:627-632, Whitt et al. (1991) Virology 185:681-688, Dietzschold et al. (1978) J. Gen. Virol. 40:131-139, Dietzschold et al. (1978) Dev. Biol. Stand. 40:45-55, Dietzschold et al. (1977) J. Virol. 23:286-293, and Otvos et al. (1994) Biochim. Biophys. Acta 1224:68-76. A rabies G protein is also described in EP 0445625.

### Alternative Envelopes

Other envelopes which give reasonable titre when used to pseudotype EIAV include Mokola, Rabies, Ebola and LCMV (lymphocytic choriomeningitis virus). Following in utero injection in mice the VSV-G envelope was found to be more efficient at transducing hepatocytes than either Ebola or Mokola (Mackenzie et al. (2002)). Intravenous infusion into mice of lentivirus pseudotyped with 4070A led to maximal gene expression in the liver (Peng et al. (2001)).

### Nucleotide Sequence of Interest (NOI)

The viral vector of the present invention may be used to deliver one or more NOI(s) useful in the treatment of the disorders listed in WO 98/05635. The nucleotide sequence of interest may be DNA or RNA. For ease of reference, part of that list is now provided: cancer, inflammation or inflammatory disease, dermatological disorders, fever, cardiovascular effects, hemorrhage, coagulation and acute phase response, cachexia, anorexia, acute infection, HIV infection, shock states, graft-versus-host reactions, autoimmune disease, reperfusion injury, meningitis, migraine and aspirin-dependent anti-thrombosis; tumour growth, invasion and spread, angiogenesis, metastases, malignant, ascites and malignant pleural effusion; cerebral ischaemia, ischaemic heart disease, osteoarthritis, rheumatoid arthritis, osteoporosis, asthma, multiple sclerosis, neurodegeneration, Alzheimer's disease, atherosclerosis, stroke, vasculitis, Crohn's disease and ulcerative colitis; periodontitis, gingivitis; psoriasis, atopic dermatitis, chronic ulcers, epidermolysis bullosa; corneal ulceration, retinopathy and surgical wound healing; rhinitis, allergic conjunctivitis, eczema, anaphylaxis; restenosis, congestive heart failure, endometriosis, atherosclerosis or endosclerosis.

In addition, or in the alternative, the viral vector of the present invention may be used to deliver one or more NOI(s) useful in the treatment of disorders listed in WO 98/07859. For ease of reference, part of that list is now provided: cytokine and cell proliferation/differentiation activity; immunosuppressant or immunostimulant activity (e.g. for treating immune deficiency, including infection with human immune deficiency virus; regulation of lymphocyte growth; treating cancer and many autoimmune diseases, and to prevent transplant rejection or induce tumour immunity); regulation of haematopoiesis, e.g. treatment of myeloid or lymphoid diseases; promoting growth of bone, cartilage, tendon, ligament and nerve tissue, e.g. for healing wounds, treatment of burns, ulcers and periodontal disease and neurodegeneration; inhibition or activation of follicle-stimulating hormone (modulation of fertility); chemotactic/chemokinetic activity (e.g. for mobilizing specific cell types to sites of injury or infection); haemostatic and thrombolytic activity (e.g. for treating haemophilia and stroke); antiinflammatory activity (for treating e.g. septic shock or Crohn's disease); as antimicrobials; modulators of e.g. metabolism or behavior; as analgesics; treating specific deficiency disorders; in treatment of e.g. psoriasis, in human or veterinary medicine.

In addition, or in the alternative, the viral vector of the present invention may be used to deliver one or more NOI(s) useful in the treatment of disorders listed in WO 98/09985. For ease of reference, part of that list is now provided: macrophage inhibitory and/or T cell inhibitory activity and thus, anti-inflammatory activity; anti-immune activity, i.e. inhibitory effects against a cellular and/or humoral immune response, including a response not associated with inflammation; inhibit the ability of macrophages and T cells to adhere to extracellular matrix components and fibronectin, as well as up-regulated fas receptor expression in T cells; inhibit unwanted immune reaction and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of strokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery, bone marrow transplantation or other transplantation complications and/or side effects, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

### Therapeutic RNA

By therapeutic RNA is meant a sequence which functions at the RNA level. Preferably the therapeutic RNA does not require integration to have a therapeutic effect. More preferably the therapeutic RNA does not require reverse transcription to have a therapeutic effect.

Examples of such RNA include siRNA, shRNA, micro-RNA, or regulated sh or micro RNA (Dickins et al. (2005) Nature Genetics 37:1289-1295; Silva et al. (2005) Nature Genetics 37:1281-1288) a ribozyme, an mRNA or a tRNA. The vector particle may also be used to deliver an antisense sequence.

Post-transcriptional gene silencing (PTGS) mediated by double-stranded RNA (dsRNA) is a conserved cellular defense mechanism for controlling the expression of foreign genes. It is thought that the random integration of elements such as transposons or viruses causes the expression of dsRNA which activates sequence-specific degradation of homologous single-stranded mRNA or viral genomic RNA. The silencing effect is known as RNA interference (RNAi) (Ralph et al. (2005): Nature Medicine 11:429-433). The mechanism of RNAi involves the processing of long dsRNAs into duplexes of about 21-25 nucleotide (nt) RNAs. These products are called small interfering or silencing RNAs (siRNAs) which are the sequence-specific mediators of mRNA degradation. In differentiated mammalian cells dsRNA >30 bp have been found to activate the interferon response leading to shut-down of protein synthesis and non-specific mRNA degradation (Stark et al. (1998)). However this response can be bypassed by using about 21 nt siRNA duplexes (Elbashir et al. (2001), Hutvagner et al. (2001)) allowing gene function to be analysed in cultured mammalian cells.

In another embodiment the NOI comprises a micro-RNA. Micro-RNAs are a very large group of small RNAs produced naturally in organisms, at least some of which regulate the expression of target genes. Founding members of the micro-RNA family are let-7 and lin-4. The let-7 gene encodes a small, highly conserved RNA species that regulates the expression of endogenous protein-coding genes during worm development. The active RNA species is transcribed initially as a ˜70 nt precursor, which is post-transcriptionally processed into a mature ˜21nt form. Both let-7 and lin-4 are transcribed as hairpin RNA precursors which are processed to their mature forms by Dicer enzyme.

### Transient Expression

It may be desirable, in a therapeutic setting, to be able to transiently express proteins or transiently knock-down expression of proteins. This may be achieved by delivering non-integrating viral vectors to target cells. Preferably the non-integrating vectors are unable to undergo reverse transcription from RNA to DNA. RNA has a finite lifetime in cells and once it has been degraded the phenotype it conferred on cells would be removed. This has been difficult to achieve to date because a lack of suitable vectors and transfection methods for RNA have yet to achieve satisfactory levels of transfer in vivo.

The viral constructs of the present invention can be used to specifically package therapeutic RNA for efficient delivery in vivo.

A preferred NOI for use in the present invention is a therapeutic RNA. By therapeutic RNA it is meant a sequence which functions at the RNA level. Preferably the therapeutic RNA does not require integration to have a therapeutic effect. More preferably the therapeutic RNA does not require reverse transcription to have a therapeutic effect. Preferably the therapeutic RNA is a catalytic RNA.

### Promoters

Expression of a NOI may be controlled using control sequences, which include promoters/enhancers and other expression regulation signals. Prokaryotic promoters and promoters functional in eukaryotic cells may be used. Tissue specific or stimuli specific promoters may be used. Chimeric promoters may also be used comprising sequence elements from two or more different promoters.

Suitable promoting sequences are strong promoters including those derived from the genomes of viruses—such as polyoma virus, adenovirus, fowlpox virus, bovine papilloma virus, avian sarcoma virus, cytomegalovirus (CMV), retrovirus and Simian Virus 40 (SV40)—or from heterologous mammalian promoters—such as the actin promoter or ribosomal protein promoter. Transcription of a gene may be increased further by inserting an enhancer sequence into the vector. Enhancers are relatively orientation and position independent; however, one may employ an enhancer from a eukaryotic cell virus—such as the SV40 enhancer on the late side of the replication origin (bp 100-270) and the CMV early promoter enhancer. The enhancer may be spliced into the vector at a position 5′ or 3′ to the promoter, but is preferably located at a site 5′ from the promoter.

The promoter can additionally include features to ensure or to increase expression in a suitable host. For example, the features can be conserved regions e.g. a Pribnow Box or a TATA box. The promoter may even contain other sequences to affect (such as to maintain, enhance, decrease) the levels of expression of a nucleotide sequence. Suitable other sequences include the Sh1-intron or an ADH intron. Other sequences include inducible elements—such as temperature, chemical, light or stress inducible elements. Also, suitable elements to enhance transcription or translation may be present.

### Pharmaceutical Compositions

The present invention also provides a pharmaceutical composition for treating an individual by gene therapy, wherein the composition comprises a therapeutically effective amount of the retroviral or lentiviral vectors of the present invention comprising one or more deliverable therapeutic and/or diagnostic NOI(s) or a viral particle produced by or obtained from same. The pharmaceutical composition may be for human or animal usage. Typically, a physician will determine the actual dosage which will be most suitable for an individual subject and it will vary with the age, weight and response of the particular individual.

The composition may optionally comprise a pharmaceutically acceptable carrier, diluent, excipient or adjuvant. The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilizing agent(s), and other carrier agents that may aid or increase the viral entry into the target site (such as for example a lipid delivery system).

Where appropriate, the pharmaceutical compositions can be administered by any one or more of: inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavoring or coloring agents, or they can be injected parenterally, for example intracavernosally, intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration, the compositions may be administered in the form of tablets or lozenges which can be formulated in a conventional manner.

### Treatment

It is to be appreciated that all references herein to treatment include curative, palliative and prophylactic treatment. The treatment of mammals is particularly preferred. Both human and veterinary treatments are within the scope of the present invention.

The present invention will now be further described by way of the following non-limiting examples, provided for illustrative purposes only.

### EXAMPLES

### Example 1

### Protection from Staurosporine (STS)-Mediated Toxicity in Cortical Neurons Transduced with Integrase Defective Vectors

Primary cortical neurons were cultured from Wistar rat embryos (gestation day 18) and plated at a density of 75,000 cells per well of a 24-well plate. One week post plating cells were transduced with EIAV vectors (using a multiplicity of infection of 10 transducing units/cell) encoding the anti-apoptotic gene BCL-2 or a control vector. A third EIAV vector encoding the BCL-2 transgene, but lacking the gene responsible for mediating integration of the viral genome into the host cell (integrase minus), was also transduced. Five days post transduction the cells were exposed to staurosporine at a concentration of 1 μM for 24 hours. Cells were assessed for viability using the MTT assay and induction of apoptosis was investigated using a caspase 3/7 detection kit (Promega). The MTT assay demonstrated that over expression of BCL-2 from the integrase positive EIAV vector mediated significant protection from staurosporine-mediated toxicity compared with the control vector. Furthermore, the integrase minus vector encoding BCL-2 also conferred a similar neuroprotective effect. Analysis of caspase 3/7 activation demonstrated that both the integrase positive and integrase negative EIAV vectors encoding BCL-2 mediated significant reduction in caspase 3/7 activation following treatment with staurosporine compared with an EIAV vector control.

### Example 2

### Conditioned Medium from Cortical Neurons Transduced with BCL-2-Flag is Unable to Protect Against Staurosporine-Induced Apoptosis

To investigate whether the neuroprotective effect described above was a consequence of BCL-2 secretion into the culture media or BCL-2 over expression mediating the release of some other survival factor, conditioned media from similar cultures to those described above, and transduced for 5 days with the same EIAV vectors, were removed and placed onto untransduced sister cultures. The cells were exposed to 1 μM staurosporine for 24 hours and the MTT assay performed immediately after the incubation period. The results of the MTT assay demonstrated that the conditioned media from each of the EIAV transduced cultures did not mediate any neuroprotection against staurosporine-induced toxicity. This result suggests that the results observed above were not mediated by a released neuroprotective factor or secreted BCl-2 from transduced cells.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

Having thus described in detail preferred embodiments of the present invention, it is to be understood that the invention defined by the appended claims is not to be limited to particular details set forth in the above description, as many apparent variations thereof are possible without departing from the spirit or scope of the present invention. Modifications and variations of the method and apparatuses described herein will be obvious to those skilled in the art, and are intended to be encompassed by the following claims.

1-49. (canceled)

50. An integration defective retroviral vector particle comprising a nucleotide sequence encoding a disabled integrase protein, wherein the disabled integrase protein comprises a DDE motif that is absent or replaced.

51. The integration defective retroviral vector particle of claim 50, wherein each of the D, D, and E amino acids of the DDE motif is replaced or absent from the disabled integrase protein.

52. The integration defective retroviral vector particle of claim 50, wherein the retroviral vector particle is a lentiviral vector particle.

53. The integration defective retroviral vector particle of claim 50, wherein the integration defective retroviral vector particle comprises a retroviral vector genome comprising a disabled primer binding site (PBS) and/or att site.

54. The integration defective retroviral vector of claim 53, wherein the retroviral vector particle comprises a disabled reverse transcriptase.

55. The integration defective retroviral vector of claim 50, wherein the integration defective retroviral vector particle comprises a retroviral vector genome comprising an NOI.

56. The integration defective retroviral vector of claim 55, wherein the NOI is an RNA sequence selected from the group consisting of mRNA, shRNA, siRNA, microRNA, ribozyme and tRNA.

57. An integration defective retroviral vector particle comprising a nucleotide sequence encoding a disabled integrase protein, wherein nucleotide sequences encoding a functional integrase protein are absent.

58. The integration defective retroviral vector particle of claim 57, wherein nucleotide sequences corresponding to the entire integrase gene are absent.

59. The integration defective retroviral vector particle of claim 57, wherein the retroviral vector particle is a lentiviral vector particle.

60. An integration defective retroviral vector production system, comprising a set of nucleic acid sequences encoding a retroviral vector genome, gag, pol, and an envelope, wherein the nucleic acid sequences comprise a nucleotide sequence encoding a disabled integrase protein, wherein the disabled integrase protein comprises a DDE motif that is absent or replaced, and wherein the integration defective retroviral vector production system is capable of producing an integration defective retroviral vector particle.

61. The integration defective retroviral vector production system of claim 60, wherein each of the D, D, and E amino acids of the DDE motif is replaced or absent from the disabled integrase protein.

62. The integration defective retroviral vector production system of claim 60, wherein the integration defective retroviral vector production system is an integration defective lentiviral vector production system.

63. The integration defective retroviral vector production system of claim 60, wherein the retroviral vector genome comprises a disabled primer binding site (PBS) and/or att site.

64. The integration defective retroviral vector production system of claim 63, wherein the retroviral vector genome comprises a disabled reverse transcriptase.

65. The integration defective retroviral vector production system of claim 60, wherein the set of nucleic acid sequences comprising an NOI.

66. The integration defective retroviral vector production system of claim 65, wherein the NOI is an RNA sequence selected from the group consisting of mRNA, shRNA, siRNA, microRNA, ribozyme and tRNA.

67. A method of transducing a cell with an integration defective retroviral vector according to claim 50.
